(19) United States
(12) Patent Application Publication
(10) Pub. No.: US 2013/0040849 A1
(43) Pub. Date: Feb. 14, 2013

(54) METHOD AND KIT FOR CANCER DIAGNOSIS

(71) Applicants: Rune Eliasson, Hasselby (SE); Nils Egberg, Lidingo (SE); Lena Carlsson, Uppsala (SE); Gunnar Ronquist, Uppsala (SE); Anders Larsson, Uppsala (SE); Goran Ronquist, Uppsala (SE)

(72) Inventors: Rune Eliasson, Hasselby (SE); Nils Egberg, Lidingo (SE); Lena Carlsson, Uppsala (SE); Gunnar Ronquist, Uppsala (SE); Anders Larsson, Uppsala (SE); Goran Ronquist, Uppsala (SE)

(73) Assignee: PROSTASOM HANDELSBOLAG, HASSELBY (SE)

(21) Appl. No.: 13/641,424

(22) PCT Filed: Apr. 18, 2011
(86) PCT No.: PCT/SE2011/050468

§ 371 Date: Oct. 29, 2012

Related U.S. Application Data
(60) Provisional application No. 61/324,914, filed on Apr. 16, 2010.

(30) Foreign Application Priority Data
Apr. 16, 2010 (SE) 1050373-8

(51) Int. Cl. G01N 33/574; G01N 21/64; C07K 16/28; C40B 30/04

(52) U.S. Cl. 506/9; 435/7.23; 530/389.1

(57) ABSTRACT

A method for diagnosing or providing a prognosis of a subject suspected of suffering from prostate cancer, comprising in vitro detection of prostasomes and quantification of prostasomal expression of at least one antigen chosen from the group consisting of CD13, CD59, CD10, CD26 CD142, CD143 and MHC I, and comparing said quantified expression value with a reference value for the respective antigen derived from healthy subject(s). Quotients between said antigens may moreover be made use of. Detection may be by way of flow cytometry or ELISA. A kit for use in diagnosis or providing a prognosis of a subject suspected of suffering from prostate cancer is furthermore provided.

### FIELD OF INVENTION

The present invention relates to a method and a kit for use in the diagnosis or providing a prognosis of a subject suspected of suffering from prostate cancer.

### BACKGROUND OF THE INVENTION

Cancer is one of the most prevalent deadly diseases, which despite recent advances in diagnosis and treatment still accounts for a substantial number of deaths each year.

Prostate cancer, as an example, is the most prevalent cancer disease of ten diagnosed cancer diseases in men exhibiting symptoms derived from a local tumour or metastatic spread of a tumour, such as dysfunctional voiding or bone pain, and the disease is often at an advanced stage at the time of diagnosis. Occasionally, it is an accidental finding on digital rectal examination or upon histological examination of tissue obtained during surgery on men with benign prostatic hyperplasia.

Measurement of prostate specific antigen (PSA) has changed the pattern of diagnosis of prostate cancer with more cases detected at an early stage and fewer cases at advanced stages. However, since PSA is not a prostate cancer specific marker in serum it is not the ideal diagnostic marker and therefore not accommodated for screening of prostate cancer. The PSA test cannot discriminate between benign prostatic hyperplasia and prostate cancer at an ealy stage and, what is more, between prostate cancer with high metastasising potential (aggressive prostate cancer) and such cancer with no or weak aggressiveness (indolent prostate cancer). An “unspecified” PSA increase most often leads to multiple biopsies of the prostate, that in many cases is a disadvantage for the patient and society alike. Inter alia, the PSA test bears the risk that the patient may acquire blood poisoning due to the use of biopsy during follow-up examination.

The shortcomings of the PSA test moreover often result in truncating surgery, i.e. total prostatectomy. Overtreatment of this kind is a great inconvenience not only for patients but also for society due to extra costs.

Prostasomes, as a group of exosomes, are secretory products of the prostate gland. The membrane architecture of these organelles is complex and two-dimensional gel electrophoresis of membrane material has revealed more than 100 different protein entities. The prostasomes contain neuroendocrine and CD (cluster of differentiation) molecules and many different enzymes are part of the prostasome membrane mosaic. Prostasomes have been ascribed many different biological activities, but their physiologic function is still unclear.

In WO2007/015174, exosome-specific ligands and compositions comprising the same are disclosed. U.S. Pat. No. 7,083,796 discloses composititions and fusion proteins containing at least Mycobacterium sp. antigens and RNA encoding such compositions and fusion proteins. In U.S. Pat. No. 6,620,922 it is disclosed compostions and methods for the therapy and diagnosis of cancer, such as prostate cancer. U.S. Pat. No. 6,107,090 discloses the use of antibodies or binding portions thereof, probes, ligands, or other biological agents which either recognize an extracellular domain of prostate specific membrane antigen or bind to and are internalized with prostate specific membrane antigen. In “Perspectives on the Biological Role of Human Prostasomes” Lena Carlsson, Doctoral Thesis 2001 it is disclosed that prostasomes have potent anti-bacterial effect. In “Flow cytometric technique for determination of prostasomal quantity, size and expression of CD10, CD13, CD26 and CD59 in human seminal plasma”, Lena Carlsson et al, international journal of andrology (2006) the prostasomal expression of CD markers is discussed. As follows from the above stated, there is a need for improved, non-invasive diagnostic tools for the detection of prostate cancer. The aforementioned publications, the contents of which being included herein to the greatest extent permitted by law, do not disclose any non-invasive diagnostic tool for the detection of prostate cancer.

### DESCRIPTION OF THE INVENTION

According to a first aspect of the invention, there is provided a method for diagnosing or providing a prognosis of a subject suspected of suffering from prostate cancer, comprising in vitro detection of prostasomes and quantification of prostasomal expression of at least one antigen and comparing said quantified expression value with a reference value for the respective antigen derived from healthy subject(s). Healthy subject(s) is (are) defined herein as subject(s) not having subjective and/or clinical signs of cancer e.g. not having prostate cancer.

The in vitro detection of prostasomes as a new type of biomarkers and quantification of prostasomal expression of at least one antigen, respectively, may be performed in one single or in two individual steps. Antibodies used for the in vitro detection of prostasomes may be the same as the antibodies used in the subsequent quantification of prostasomal antigen expression, or different. By way of example, the monoclonal antibodies mAb78 (see e.g. Floryk D et al., Differentiation of human prostate cancer PC-3 cells induced by inhibitors of inosine 5″-monophosphate dehydrogenase. Cancer Res. 2004; 64: 9049-9056) and mAb8H10 have been found to be useful for the in vitro detection of prostasomes.

In a step prior to the detection and quantification of prostasomal expression, a sample from the subject suspected of suffering from prostate cancer is provided. The method of the invention is non-invasive, i.e. no biopsies are needed. The sample may be a body fluid, such as prostate secretion, urine, seminal fluid, blood, in natural or processed form (see Table 4). Processing of the sample may be e.g. centrifugation.

According to one embodiment of the invention, the sample is enriched for prostasomes between the provision of said sample and the subsequent step of detection and quantification of the prostasomal antigen expression. This enrichment may be carried out by immobilizing the prostasomes on a solid support, e.g. on nitrocellulose membranes, in radioimmunoassay tubes, on particles such as nanoparticles, or on ELISA plates.

In one embodiment of the invention, the at least one antigen is chosen from the group consisting of CD13, CD59, CD10, CD26 CD142, CD143 and MHC I.

In one embodiment of the invention, the method of diagnosing or providing a prognosis of a subject suspected of suffering from prostate cancer is based on up-regulation of at least one of antigens CD10, CD26, CD142 (also known as Tissue Factor) and MHC I in subjects suffering from prostate cancer, compared with the reference value. Accordingly, in accordance with the invention, the quotient between the reference value (i.e. control) and CD26 expressed on prostasomes from prostate cancer patients may be 0.95 or less. Likewise, for CD142 the quotient between the reference value (i.e. control) and CD142 expressed on prostasomes from prostate cancer patients may be 0.75 or less. For MHC I, the quotient between the reference value (i.e. control) and MHC I expressed on prostasomes from prostate cancer patients may also be 0.75 or less, for prostate cancer to be diagnosed or prognosticated.

In an alternative embodiment, the method of diagnosing or providing a prognosis of a subject suspected of suffering from prostate cancer is based on down-regulation of at least one of antigens CD13 and CD59 in subjects suffering from prostate cancer, compared with the reference value. Accordingly, in accordance with the invention, the quotient between the reference value (i.e. control) and CD13 expressed on prostasomes from prostate cancer patients may be 1.59 or more. Likewise, for CD59, the quotient between the reference value (i.e. control) and CD59 expressed on prostasomes from prostate cancer patients shall be 1.30 or more.

The reference value made use of may derive from healthy subjects confirmed not to suffer from prostate cancer by the use of PSA, clinical and anamnestic data.

The expression of the above mentioned antigens may be measured by measuring antibodies reacted with the antigens. The antibodies may be labeled for detection. The detection may be performed e.g. with fluorescence e.g. in flow cytometry and be evaluated with ROC curves.

The person skilled in the art realizes that the mean fluorescence intensities on which the above quotients are calculated may vary with the analytical equipment employed. It is likely, however, that the quotients would to a great extent remain unaltered. Likewise the skilled person realizes that other methods may be used for measuring the expression of the antigens. Again the above mentioned quotients would to a great extent remain unaltered.

In one embodiment of the invention, a quotient is calculated between at least two of the antigens and the quotient is compared with a reference quotient value. A quotient may be based on e.g. down-regulated prostasomal antigens from prostate cancer patients divided by up-regulated prostasomal antigens from the same prostate cancer patient. This quotient value is then compared with a reference quotient value from normal patients without prostate cancer. The use of such a quotient may give the method improved prognostic value.

Consequently, in one embodiment of the invention, the quotient (CD13+CD59)/(CD10+CD26) is determined, based on the amounts of the antigens detected, and compared with a reference value. CD13 and CD59 are both down-regulated on prostasomes from prostate cancer patients, whereas CD10 and CD26 are both up-regulated on prostasomes from prostate cancer patients. The person skilled in the art realizes that quotients may be calculated and be made use of using also other combinations of antigens than the one combination specifically disclosed herein.

In a further embodiment of the current invention, at least one kind of antibodies capable of binding specifically to at least one of the antigens are used to detect and quantify the expression thereof. The antibodies may be monoclonal or polyclonal. As an alternative to intact antibodies, Fab, Fab2 or single chain Fv antibodies may be made use of. The intact antibodies or parts thereof shall all possess the relevant antigen binding domain.

In yet an embodiment of the invention, the at least one kind of antibodies are labeled with distinguishable fluorescent marker(s). Use may be made of flow cytometry for detecting and quantifying said antibodies to get an expression pattern. In an alternative embodiment, ELISA is made use of for detecting and quantifying said antibodies. DELPHIA analysis using e.g. europium and samarium may also be utilized in accordance with the invention.

According to a second aspect of the invention, there is provided a kit for use in diagnosis or providing a prognosis of a subject suspected of suffering from prostate cancer, comprising at least one kind of antibodies specifically binding to specifically to at least one antigen chosen from the group consisting of CD13, CD59, CD10, CD26, CD142, CD143 and MHC I. Said antibodies may be monoclonal or polyclonal, or alternatively Fab, Fab2 or single chain Fv antibodies may form part of the kit.

The invention is further described in the following examples in conjunction with the appended figures, which are not intended to limit the scope of the invention in any way whatsoever.

### SHORT DESCRIPTION OF THE FIGURES

FIG. 1 shows the ROC curve for the ratio between CD13 and CD10. The AUC (area under the curve) for distinguishing prostate cancer patients from normal individuals (without prostate cancer) was 0.874 and the ratio had a sensitivity of 83.3% and a specificity of 91.1% at a cut off limit of 2.04

FIG. 2 shows the ROC curve for the ratio between CD59 and CD10. The AUC for distinguishing prostate cancer patients from normal individuals (without prostate cancer) was 0.863 and the ratio had a sensitivity of 100% and a specificity of 64.6% at a cut off limit of 5.32

### EXAMPLES

### Sample Preparation

Seminal samples from 79 patients without known prostate cancer according to PSA values and clinical and anamnestic information and 10 patients with confirmed prostate cancer were provided.

Seminal fluids were obtained from human semen after incubation thereof at room temperature for about 30 min and centrifugation for 20 min at 1000×g at a temperature of 20° C., in order to separate sperms from seminal plasma. The seminal plasma was thereby ready for the flow cytometry analysis described below.

Urine, prostate secretion and heparinized blood samples, respectively, were centrifuged at 2500×g for 10 min at a temperature of 20° C. The supernatants from prostate secretion, urine and heparinized blood, respectively, were ready for analysis with flow cytometry. The blood plasma obtained by centrifugation was moreover tested on solid phase with ELISA and dot blot/immune blot on nitrocellulose paper with specific antibodies against prostasomes (see ELISA and immunoblotting sections below).

### Preparation of Samples for Flow Cytometry

30 μL of diluted seminal plasma or of the respective supernatant (each diluted 1:25 in Phosphate Buffered Saline (PBS)) were added to polystyrene tubes containing 10 μL FITC-labeled antibodies. Each antibody was added to a separate tube and thus each marker was subsequently analyzed separately.

The following monoclonal antibodies, capable of binding to the extracellular domain of the respective protein, were used (all antibodies obtained from Serotec (Kidlington, UK):

FITC-CD10 (antibody MCA1556F, 0.1 mg/mL),
FITC-CD13 (antibody MCA1270F, 0.1 mg/mL),
FITC-CD26 (antibody MCA1317F, 0.1 mg/mL),
FITC-CD59 (antibody MCA1054F, 0.1 mg/mL),
FITC-CD142 (antibody MCA2548F, 0.1 mg/mL),
FITC CD143 (antibody MCA2057F, 0.1 mg/mL)

The samples were incubated for 10 min at 20° C. and were then analyzed by flow cytometry. No washing steps were performed.

### Flow Cytometry

Samples were analyzed utilising an Epics Profile XL-MCL cytometer (Coulter Electronics, Hialeah, Fla.). The flow cytometer detects individual cells or organelles and present them in a regular manner in a scatter plot. 100 μL of each sample was analyzed for determination of prostasome concentration and size. For analyses of CD markers, data processing of 5,000 prostasomes was carried out using the XL software (Coulter Electronics), for each individual patient. Based on light scattering properties, each prostasome was represented by a point in a rectangular co-ordinate system. The location and size of the gate was set taking into account that the prostasomes were highly purified. Discrimination frames were placed around the prostasome cluster and the flow count cluster using forward and side scatter.

The flow cytometer measures the fluorescent signal from labeled antibodies bound to the prostasomes. The flow cytometer gives percentage of positively stained prostasomes, median and mean fluorescence intensity (MFI), complexity (right angle scatter), and median and mean size (forward angle scatter) of the prostasome population within the field. Analytical markers were set in the fluorescence channel to measure MFI (wavelength=225 nm for FITC). The gates were set prior to the study.

### Flow Cytometry on Seminal Fluid

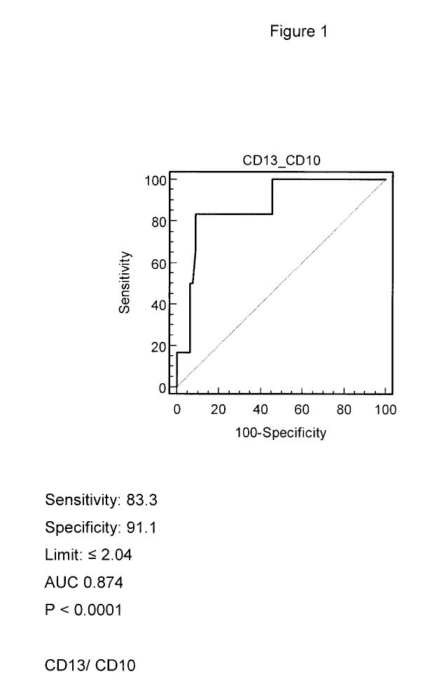

Samples from prostate cancer patients displayed a diverging protein pattern in comparison with controls. The sensitivity and specificity of the markers were evaluated with ROC curves. The markers CD13, CD59 and the ratios CD13/CD10 and CD59/CD26, respectively, were the markers that gave the highest sensitivity and specificity for prostate cancer. CD13/CD10 yielded the highest specificity (91.1%, FIG. 1) while CD59/CD26 yielded the highest sensitivity (100%). Particles with side and forward scatter typical for prostasomes were detected.

### Flow Cytometry on Serum and Urine

10 μL of seminal plasma from cancer patients and controls were added to polystyrene tubes containing 100 μL serum or urine and the samples were diluted 1:25 with PBS. 30 μL of the diluted sample and 10 μL of FITC-labeled antibodies were mixed and the mixtures were incubated for 10 min at a temperature of 20° C. and were then analysed using flow cytometry. No washing steps were performed.

100 μL of serum from two patients with high PSA was also analyzed as above but without addition of purified prostasomes. Particles with side and forward scatter typical for prostasomes were detected.

### Flow Cytometry Results (Serum and Urine)

The CD expression of prostasomes re-suspended in serum or urine was undisturbed with unaltered results in comparison with the original seminal fluid. The patients sample showed organelles with a forward and side scatter representative of prostasomes. The particles were CD13 positive showing that the particles were prostasomes (see Table 4).

### Preparation of Purified Prostasomes

Purified prostasomes were needed to set the discrimination gates of the flow cytometer. Seminal plasma samples were pooled (12-15 samples) and ultracentrifuged at 10 000×g at 4° C. for 15 min to remove possible cell debris. The supernatant was then subjected to another ultracentrifugation for 2 h at 100 000×g at 4° C. to pellet the prostasomes. The prostasomes were resuspended in isotonic Tris-HCl buffer, pH 7.6, and further purified on a Sephadex G 200 gel column (Amersham Biosciences, Uppsala, Sweden). The eluate was monitored at 260/280 nm. Those fractions with elevated UV absorbances were collected and analyzed for CD13, a strong marker enzyme for prostasomes. Ultraviolet-absorbing fractions with high CD13 activity were pooled and ultracentrifuged at 100 000×g at 4° C. for 2 h. The pellet representing the purified prostasomes was resuspended in isotonic Tris-HCl buffer, pH 7.6, and adjusted to a suitable protein concentration.

### Immunoblotting

1 μL of purified seminal prostasomes was pipetted onto a nitrocellulose membrane and the membrane was blocked with 1% BSA in 0.02 M Na₂HPO₄, 0.15 M NaCl, pH 7.2 (PBS) for 1 hour. After 3 washes the membrane was incubated with 100 μL of biotinylated polyclonal chicken anti-prostasomal antibodies (diluted 1:1000 in PBS with 1% BSA) for 1 h at 20° C. After 3 new washes with PBS-T (PBS with 0.05% Tween 20), 100 μL Streptavidine-alkaline phosphatase conjugate (Zymed Laboratories, Inc., CA, USA), diluted 1:2,000 in PBS with 1% BSA, was added and incubated for 1 h at 20° C. After three washes, the nitrocellulose membrane was developed.

### Immunoblotting Result

The antibody recognized the prostasomes on the nitrocellulose membrane and a purple dot was visualized. This shows that the prostasomes can be captured on a solid phase (in this case a nitrocellulose membrane) and then detected by antibodies.

### ELISA

Microtitre plates (F96, Polysorp, Nunc) were coated with 4 μg/mL of purified seminal prostasomes in 0.1 M NaHCO₃, pH 9.5 for 2 h at 20° C. The plates were washed 3 times with 0.02 M Na₂HPO₄, 0.15 M NaCl, 0.05% Tween 20, pH 7.2 (PBS-T) and the plates were blocked with 1% BSA in 0.02 M Na₂HPO₄, 0.15 M NaCl, pH 7.2 for 2 hours. After 3 washes the plates were incubated with 100 μL of polyclonal chicken anti-prostasomal antibody (diluted 1:1000 in PBS) for 2 h at 20° C. After 3 new washes with PBS-T, 100 μL goat anti-chicken IgG horseradish peroxidase (HRP)-conjugated antibodies (Zymed Laboratories, Inc., CA, USA), diluted 1:2,000 in PBS, were added and incubated for 2 h at 20° C. The plates were washed 3 times with 250 μL PBS-T and incubated with substrate (tetramethyl benzidine, Zymed Laboratories, Inc.) for 15 min at 20° C. while being protected from light. The reaction was stopped by adding 50 μL of 1.8 mol/L sulphuric acid. The absorbance was measured at 450 nm in an ELISA reader system (SPECTRA Max 250, Molecular Devices, Sunnyvale, Calif., USA).

### ELISA Results

The anti-prostasome antibody (Immunsystem AB, Uppsala, Sweden) gave a positive reaction with the prostasomes bound to the ELISA plate with an absorbance value of >1.0. This shows that the prostasomes can be captured on a solid phase (in this case a microtiter plate) and then detected by antibodies, such as enzyme labeled antibodies as in this case.

### Experimental Results

Below, comparative results between controls and patients suffering from prostate cancer are presented (Tables 1, 2 and 3). Moreover, Table 4 shows measurements on seminal fluid, serum and urine, respectively.

TABLE 1

| | CD10 | CD13 | CD26 | CD59 | MHCl |
|---|---|---|---|---|---|
| | MFI | MFI | MFI | MFI | MFI |
| Control | | | | | |
| 1 | 1.3 | 2.3 | 3.4 | 4 | 1.4 |
| 2 | 1.9 | 4 | 1.9 | 10.5 | 1.8 |
| 3 | 1.3 | 3.4 | 1.3 | 10.2 | 2.9 |
| 4 | 0.74 | 2.8 | 0.78 | 6.3 | 0.9 |
| 5 | 2.1 | 5.3 | 2.2 | 10.7 | 0.7 |
| 6 | 3.1 | 8 | 2.5 | 21.7 | 1.1 |
| 7 | 2.8 | 8.6 | 3.8 | 17.5 | 1.7 |
| 8 | 1.5 | 4.7 | 1.4 | 9.5 | 1.2 |
| 9 | 2.5 | 4.6 | 2.2 | 12.7 | 1.8 |
| 10 | 1.4 | 2.8 | 1.6 | 7.2 | 0.6 |
| 11 | 1.1 | 1.3 | 1.3 | 3.6 | 1.9 |
| 12 | 0.9 | 2.6 | 0.9 | 5.6 | 2.2 |
| 13 | 1.7 | 4 | 2.1 | 8.1 | 2.8 |
| 14 | 2.1 | 5.8 | 2.1 | 12.4 | 1.2 |
| 15 | 1.7 | 6.4 | 2.3 | 16 | 1 |
| 16 | 1.2 | 2.7 | 1.8 | 5.5 | 1.5 |
| 17 | 1.5 | 4 | 1.4 | 10.2 | 2.9 |
| 18 | 1.5 | 5.8 | 1.8 | 12 | 2.4 |
| 19 | 1.9 | 4.7 | 2.1 | 8.8 | 0.6 |
| 20 | 1.8 | 5.6 | 2.2 | 8.2 | 0.6 |
| 21 | 1.7 | 4 | 2.1 | 9.5 | 2.4 |
| 22 | 1.7 | 3.5 | 2.1 | 8.1 | 1.7 |
| 23 | 1.9 | 4.4 | 2 | 13 | 1.2 |
| 24 | 1.4 | 5.3 | 1.6 | 11 | 1.9 |
| 25 | 2.5 | 6.5 | 2.4 | 9.2 | 1.3 |
| 26 | 1.6 | 4.1 | 2.1 | 11.9 | 2.9 |
| 27 | 0.8 | 3.8 | 1.3 | 5.4 | 3.1 |
| 28 | 1.5 | 5.9 | 2.7 | 10.9 | 2.4 |
| 29 | 2.5 | 8.5 | 2.6 | 15.9 | 2.7 |
| 30 | 1.4 | 5.3 | 1.6 | 11 | 1.6 |
| 31 | 2.1 | 8 | 3.2 | 16.8 | 1.3 |
| 32 | 1.3 | 5.3 | 1.8 | 10.2 | 1.9 |
| 33 | 1.4 | 3.7 | 1.3 | 10.9 | 1.2 |
| 34 | 1.7 | 3.4 | 2.2 | 8 | 1.1 |
| 35 | 1.2 | 1.6 | 1.6 | 4 | 0.5 |
| 36 | 1.4 | 3.8 | 1.3 | 9.2 | 1.9 |
| 37 | 2.1 | 6.5 | 2.8 | 12.4 | 1.3 |
| 38 | 1.5 | 4.7 | 2 | 10.6 | 1.6 |
| 39 | 2.1 | 6.2 | 3.8 | 16 | 0.8 |
| 40 | 1.5 | 4.7 | 1.4 | 9.5 | 0.9 |
| 41 | 1.8 | 4 | 1.6 | 11.3 | 2.7 |
| 42 | 1.4 | 2.8 | 1.6 | 7.2 | 2.6 |
| 43 | 2.1 | 6.5 | 3.5 | 13.1 | 2.8 |
| 44 | 0.94 | 1.3 | 0.9 | 4 | 1.7 |
| 45 | 1.9 | 8.2 | 1.6 | 16.2 | 1 |
| 46 | 1.3 | 3.5 | 1.6 | 9.2 | 1.1 |
| 47 | 0.87 | 2 | 1.6 | 4.2 | 1.8 |
| 48 | 1.8 | 6.6 | 2.3 | 12.6 | 0.9 |
| 49 | 1.1 | 1.5 | 2.2 | 6.1 | 0.6 |
| 50 | 1.7 | 4 | 2.1 | 8.1 | 0.4 |
| 51 | 2.1 | 5.8 | 2.1 | 12.4 | 1.2 |
| 52 | 1.7 | 6.4 | 2.3 | 16 | 1.7 |
| 53 | 1.2 | 2.7 | 1.8 | 5.5 | 2.8 |
| 54 | 1.5 | 4 | 1.4 | 10.2 | 2.5 |
| 55 | 1.5 | 5.8 | 1.8 | 12 | 2 |
| 56 | 1.4 | 7.6 | 1.4 | 14.7 | 1.3 |
| 57 | 1.9 | 10.6 | 1.9 | 12.9 | 1.2 |
| 58 | 1.8 | 8.3 | 1.6 | 16.3 | 0.7 |
| 59 | 2.1 | 5.4 | 2.1 | 11.1 | 0.5 |
| 60 | 1.1 | 1.5 | 2.1 | 6.1 | 0.6 |
| 61 | 1.9 | 8.8 | 1.6 | 16.2 | 1.4 |
| 62 | 1 | 1.7 | 2.3 | 3.3 | 1.1 |
| 63 | 1.2 | 3.4 | 1.3 | 9.8 | 0.8 |
| 64 | 1.2 | 2.8 | 1.3 | 8.7 | 1.8 |
| 65 | 0.94 | 3.4 | 1.1 | 7.7 | 1.2 |
| 66 | 2.1 | 5.3 | 2.2 | 10.7 | 1.9 |
| 67 | 3.1 | 8 | 2.5 | 21.7 | 1.5 |
| 68 | 1.7 | 3.4 | 1.6 | 10.4 | 0.7 |
| 69 | 1.5 | 4.7 | 1.6 | 9.3 | 2.6 |
| 70 | 1.6 | 3.9 | 1.8 | 12 | 2.7 |
| 71 | 2.9 | 6.3 | 3 | 15.2 | 3.2 |
| 72 | 1.5 | 7.3 | 1.4 | 14.1 | 2.6 |
| 73 | 1.5 | 4.7 | 1.6 | 9.3 | 1.8 |
| 74 | 1.4 | 3.7 | 1.5 | 8.2 | 1.7 |
| 75 | 1.7 | 3.6 | 1.7 | 8.7 | 1.5 |
| 76 | 2.3 | 6 | 2 | 18.7 | 0.5 |
| 77 | 1.6 | 4 | 2 | 8.1 | 0.8 |
| 78 | 1.1 | 2.6 | 1.6 | 4.6 | 1.6 |
| 79 | 1.5 | 4.6 | 2.1 | 10.1 | 1.4 |
| number | 79 | 79 | 79 | 79 | 79 |
| mean | 1.5 | 4.6 | 1.8 | 10.2 | 1.5 |
| range | 1.3-1.9 | 3.4-5.9 | 1.6-2.2 | 8.1-12.5 | 1.0-1.9 |
| Cancer | | | | | |
| patients | | | | | |
| cancerp1 | 1.3 | 2.9 | 2.4 | 7.8 | 1.9 |
| cancerp2 | 1.5 | 1.4 | 2.1 | 3.5 | 2.8 |
| cancerp3 | 1.2 | 4.1 | 1.6 | 6.1 | 2.5 |
| cancerp4 | 1.7 | 1.9 | 0.96 | 7.5 | 2.1 |
| cancerp5 | 1.6 | 2.2 | 0.89 | 8.7 | 1.8 |
| cancerp6 | 2.1 | 2.9 | 1.9 | 10.8 | 2.7 |
| cancerp7 | 2.3 | 4.7 | 3 | 11.3 | 1.3 |
| cancerp8 | 1.5 | 2.8 | 1.9 | 6.5 | 1.5 |
| cancerp9 | 1.6 | 3.3 | 3.1 | 7.8 | 1.6 |
| cancerp10 | 2.1 | 3.5 | 1.9 | 8.7 | 2.6 |
| number | 10 | 10 | 10 | 10 | 10 |
| mean | 1.6 | 2.9 | 1.9 | 7.8 | 2 |
| range | 1.5-2.0 | 2.4-3.5 | 1.7-2.3 | 6.8-8.7 | 1.6-2.6 |

TABLE 2

| | CD142 | | CD142 |
|---|---|---|---|
| Control | 1.8 | Cancerp | 2.1 |
| Control | 1.6 | Cancerp | 2.3 |
| Control | 1.7 | Cancerp | 2.1 |
| Control | 1.6 | Cancerp | 1.5 |
| Control | 2.1 | Cancerp | 2.5 |
| Control | 2.1 | Cancerp | 3.8 |
| Control | 1.7 | Cancerp | 2.8 |
| control | 1.6 | Cancerp | 2.1 |
| mean | 1.8 | mean | 2.4 |
| range | 1.6-2.1 | range | 1.5-3.8 |

TABLE 3

| | CD 10 | CD 13 | CD 26 | CD 59 | MHC |
|---|---|---|---|---|---|
| Control | 1.6 | 4.6 | 1.8 | 10.2 | 1.5 |
| Cancer | 1.5 | 2.9 | 1.9 | 7.8 | 2 |
| patient | | | | | |
| Ratio | 1.07 | 1.59 | 0.95 | 1.30 | 0.75 |
| Control/Cancer | | | | | |

TABLE 4

| | | CD13 | CD26 | MHCl |
|---|---|---|---|---|
| | | MFI | MFI | MFI |
| Cancer patient1 | Seminal | 2.8 | 1.9 | 1.5 |
| | fluid | | | |
| | serum | 2.7 | 1.9 | 1.5 |
| | urine | 2.9 | 1.8 | 1.4 |
| Cancer patient 2 | seminal | 2.9 | 2.4 | 1.9 |
| | fluid | | | |
| | serum | 2.9 | 2.4 | 1.9 |
| | urine | 2.9 | 2.4 | 1.9 |
| Cancer patient 3 | seminal | 3.3 | 3.1 | 1.6 |
| | fluid | | | |
| | serum | 3.3 | 3.1 | 1.5 |
| | urine | 3.3 | 3.1 | 1.6 |
| Control 1 | seminal | 3.4 | 1.6 | 0.7 |
| | fluid | | | |
| | serum | 3.4 | 1.5 | 0.7 |
| | urine | 3.3 | 1.5 | 0.7 |
| Control 2 | seminal | 3.4 | 1.3 | 0.8 |
| | fluid | | | |
| | serum | 3.4 | 1.3 | 0.7 |
| | urine | 3.5 | 1.3 | 0.8 |
| Control 3 | seminal | 8.3 | 1.6 | 0.7 |
| | fluid | | | |
| | serum | 8.5 | 1.6 | 0.7 |
| | urine | 8.3 | 1.5 | 0.6 |

Roc Analyses—Prognosticating Prostate Cancer Occurrence in Accordance with the Invention

The receiver operating characteristic (ROC) curve is a simple yet complete empirical description of the decision threshold effect, indicating all possible combinations of the relative frequencies of the various kinds of correct and incorrect decisions in diagnostic decision making. A ROC curve is a graphical plot of the sensitivity, or true positives, vs. (1—specificity), or false positives, for a binary classifier system as its discrimination threshold is varied. The ROC can also be represented equivalently by plotting the fraction of true positives (TPR=true positive rate) vs. the fraction of false positives (FPR=false positive rate). ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

### Results

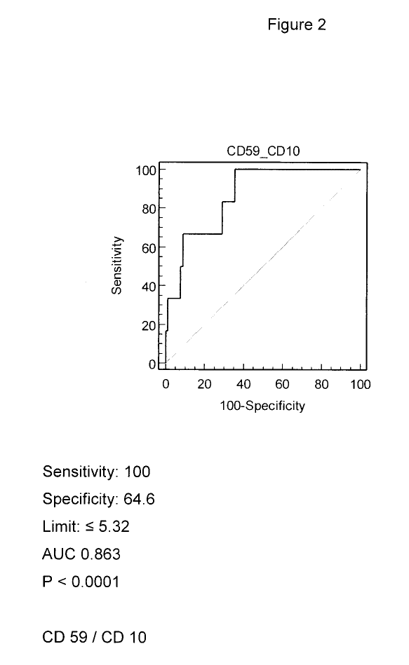

As shown in tables 1-3, prostasomes from prostate cancer patients differ in regard to the expression of the stated surface antigens. The marker antigens presented in these tables can thus be used to differentiate between prostate cancer and control subjects. The markers can be used individually or combined to improve the sensitivity and specificity of the assays (see the “Description of the Invention” above). FIG. 1 presents the ratio between CD13 and CD10 and FIG. 2 presents the ratio between CD59 and CD10 in the form of ROC curves. The sensitivity and specificity is increased compared with using only one antigen marker.

Table 4 shows that similar values were obtained when prostasomes were analyzed in various body fluids (serum, urine and seminal fluid). The flow cytometric detection of prostasomes in serum from two prostate cancer patients shows that prostasomes may also be present in serum.

Cited documents:
WO2007/015174
U.S. Pat. No. 7,083,796
U.S. Pat. No. 6,620,922
U.S. Pat. No. 6,107,090
“Perspectives on the Biological Role of Human Prostasomes” Lena Carlsson, Doctoral Thesis 2001 and
“Flow cytometric technique for determination of prostasomal quantity, size and expression of CD10, CD13, CD26 and CD59 in human seminal plasma”, Lena Carlsson et al, international journal of andrology (2005)
“Differentiation of human prostate cancer PC-3 cells induced by inhibitors of inosine 5″-monophosphate dehydrogenase” D. Floryk et al, Cancer Res. 2004; 64: 9049-9056

1. A method for diagnosing or providing a prognosis of a subject suspected of suffering from prostate cancer, comprising in vitro in vitro detection of prostasomes and quantification of prostasomal expression of at least one antigen and comparing said quantified expression value with a reference value for the respective antigen derived from healthy subject(s).

2. A method according to claim 1, wherein the at least one antigen is selected from the group consisting of CD13, CD59, CD10, CD26 CD142, CD143 and MHC I.

3. A method according to claim 2, wherein the diagnosing or providing a prognosis of a subject suspected of suffering from prostate cancer is based on up-regulation of at least one of antigens CD 10, CD26, CD 142 and MHC I in subjects suffering from prostate cancer, compared with the reference value.

4. A method according to claim 2, wherein the diagnosing or providing a prognosis of a subject suspected of suffering from prostate cancer is based on down-regulation of at least one of antigens CD13 and CD59 in subjects suffering from prostate cancer, compared with the reference value.

5. A method according to claim 1, wherein a quotient is calculated between at least two of the antigens and the quotient is compared with a reference quotient value.

6. A method according to claim 1, wherein at least one kind of antibodies capable of binding specifically to at least one of the antigens are used to detect and quantify the expression thereof.

7. A method according to claim 6, wherein the at least one kind of antibodies are labeled with distinguishable fluorescent marker(s).

8. A method according to claim 7, wherein the antibodies bound to the at least one antigen are detected and quantified by flow cytometry.

9. A method according to claim 6, wherein the antibodies bound to the at least one antigen are detected and quantified by ELISA.

10. A kit comprising at least one kind of antibodies that specifically binds to at least one antigen selected from the group consisting of CD13, CD59, CD10, CD26, CD142, CD143 and MHC I.
